(19) United States
(12) Patent Application Publication
(10) Pub. No.: US 2026/0262943 A1
(43) Pub. Date: Sep. 10, 2026

(54) SYSTEMS AND METHODS FOR SINGLE-SENSOR VISIBLE-LIGHT AND SWIR IMAGING

(71) Applicant: Cision Vision, Inc., Mountain View, CA (US)

(72) Inventor: Zhongming LI, Sunnyvale, CA (US)

(73) Assignee: Cision Vision, Inc., Mountain View, CA (US)

(21) Appl. No.: 19/167,744

(22) PCT Filed: Apr. 5, 2024
(86) PCT No.: PCT/US2024/023373

§ 371 Date: Sep. 22, 2025

Related U.S. Application Data
(60) Provisional application No. 63/495,023, filed on Apr. 7, 2023.

(51) Int. Cl. A61B 5/00; H04N 23/11; H04N 23/56

(52) U.S. Cl. CPC A61B 5/0086; A61B 5/418; H04N 23/11; H04N 23/56; A61B 5/4875; A61B 2562/0233

(57) ABSTRACT

A system for imaging biological tissue may include one or more optical sources configured to provide short-wave infrared (SWIR) illumination and to provide visible light illumination, a sensor configured to sense a reflected portion of the SWIR illumination and a reflected portion of the visible light illumination, and a controller in communication with the sensor. The controller may be configured to receive, from the sensor, first information corresponding to the reflected portion of the SWIR illumination, receive, from the sensor, second information corresponding to the reflected portion of the visible light illumination, generate at least one image of the biological tissue using the first information and the second information, and output the at least one image to at least one of a display and/or a memory.

### CROSS-REFERENCE TO RELATED APPLICATIONS

This application claims the benefit of U.S. Provisional Application No. 63/495,023, filed Apr. 7, 2023, the entire contents of which are incorporated herein by reference.

### FIELD

The present disclosure is directed generally to systems and methods for SWIR-and visible-light-based medical imaging.

### BACKGROUND

The condition of biological tissue can provide insight into the health of the person or animal to which the tissue belongs. The condition of a subject's lymph nodes, for instance, can indicate whether the subject is suffering from disease or infection. Swollen lymph nodes, for example, can be a sign of bacterial infection, viral infection, or cancer. Determining the condition of lymph nodes is therefore extremely useful for the diagnosis, prevention, and treatment of diseases.

### SUMMARY

The condition of biological tissue can be checked by imaging the tissue. Various imaging modalities exist for imaging tissue. Specialized imaging modalities can be used to image specific types of tissue. Lymph nodes, for example, can be imaged using lymphography, which involves injecting radiocontrast agents into patients and imaging the lymph nodes and lymphatic vessels via X-ray. However, lymphography is invasive, can cause significant discomfort, and requires the use of radioactive agents. Other, more general-purpose imaging modalities such computational tomography (CT), magnetic resonance imaging (MRI), ultrasound, and positron emission tomography (PET) can also be employed to image specific types of tissue. While these imaging techniques may enable biological tissue to be identified and assessed with reasonable accuracy, they may not provide ideal contrast for viewing target biological tissue unless certain contrast agents are injected. As a result, images may show organs and tissue other than the tissue of interest with the same or better contrast compared to target tissue, making finding and examining the target tissue challenging.

As described above, there exist a number of imaging modalities for visualizing and examining lymph nodes and other biological tissue. However, known medical imaging techniques have several shortcomings, for example their inability to accurately and robustly identify target tissue and/or target features and their reliance on contrast agents. Accordingly, improved medical imaging methods and systems are needed.

Provided herein are techniques for single-sensor, multi-wavelength imaging of biological tissue. The disclosed systems and methods may employ a single sensor to capture images of biological tissue in multiple wavelengths, for example to capture both short-wave infrared (SWIR) images and visible light images of the biological tissue. The captured images may be displayed, stored, and/or used to generate a composite image. Images produced using the described techniques may show target tissue (e.g., lymph nodes) and/or target features with high contrast, facilitating the identification and examination of said target tissue without requiring the use of contrast agents.

A provided system for imaging biological tissue may include one or more optical sources configured to provide short-wave infrared (SWIR) illumination and to provide visible light illumination, a sensor configured to sense a reflected portion of the SWIR illumination and a reflected portion of the visible light illumination, and a controller in communication with the sensor. The controller may be configured to receive, from the sensor, first information corresponding to the reflected portion of the SWIR illumination and to receive, from the sensor, second information corresponding to the reflected portion of the visible light illumination. Using the first information and the second information, the controller may be configured to generate at least one image of the biological tissue. The controller may be configured to then output the at least one image to at least one of a display and/or a memory.

The one or more optical sources can comprise a laser or a light-emitting diode. The sensor may comprise one or more cameras selected from the following group: a silicon camera, an InGaAs camera, a black silicon camera, a germanium camera, a germanium-tin on silicon camera, a quantum dot shortwave infrared camera, and a mercury-cadmium-telluride camera.

In some embodiments, the one or more optical sources are configured to alternately provide the SWIR illumination and the visible light illumination according to a temporal pulsing scheme. In some embodiments, the one or more optical sources comprise a first optical source configured to provide the SWIR illumination and a second optical source configured to provide the visible light illumination.

The system can further comprise a first filter configured to block at least some of the SWIR illumination and a second filter configured to block at least some of the visible light illumination. The system may include a filter support device configured to move at least one of the first filter and the second filter between a first position in an optical path of the system to a second position outside the optical path of the system.

In some embodiments, the one or more optical sources are configured to provide the SWIR illumination and the visible light illumination simultaneously. The system may further comprise a filter array configured to spatially selectively block and transmit the reflected portion of the SWIR illumination and the reflected portion of the visible light illumination. In some embodiments, the filter array comprises a first portion that transmits some or all of the reflected portion of the visible light illumination and blocks the reflected portion of the SWIR illumination, and a second portion that transmits some or all of the reflected portion of the SWIR illumination and blocks the reflected portion of the visible light illumination.

In some embodiments, the filter array comprises a first portion that transmits a green portion of the reflected visible light illumination while blocking blue and red portions of the reflected portion of the visible light illumination and blocking the reflected portion of the SWIR illumination, a second portion that transmits the blue portion of the reflected visible light illumination while blocking the green and red portions of the reflected portion of the visible light illumination and blocking the reflected portion of the SWIR illumination, a third portion that transmits the red portion of the reflected visible light illumination while blocking the blue and green portions of the reflected portion of the visible light illumination and blocking the reflected portion of the SWIR illumination, and a fourth portion that blocks the green, blue, and red portions of the reflected portion of the visible light illumination and transmits the reflected portion of the SWIR illumination. The first, second, third, and fourth portions can be arranged in a repeating 2×2 tile arrangement in the filter array or in a repeating 4×4 tile arrangement in the filter array.

If the first, second, third, and fourth portions are arranged in a repeating 4×4 tile arrangement, each repeating 4×4 tile portion in the repeating 4×4 tile arrangement may comprise twice as many green-transmissive spatial portions as it does blue-transmissive spatial portions, red-transmissive spatial portions, or SWIR-transmissive spatial portions. In some embodiments, a 4×4 tile portion does not include any laterally or vertically adjacent blue-transmissive portions, any laterally or vertically adjacent red-transmissive portions, any laterally or vertically adjacent green-transmissive portions, or any laterally or vertically adjacent SWIR-transmissive portions. In some embodiments, the 4×4 tile-arrangement comprises four 2×2 blocks, each 2×2 block comprising four spatial portions that are transmissive of a single respective wavelength range. In some embodiments, the 4×4 tile-arrangement comprises four 2×2 blocks: a first 2×2 block comprises three red-transmissive spatial subportions and one SWIR-transmissive spatial subportion, a second 2×2 block comprises three green-transmissive spatial subportions and one SWIR-transmissive spatial subportion, a third 2×2 block comprises three blue-transmissive spatial subportions and one SWIR-transmissive spatial subportion, and a fourth 2×2 block comprises a red-transmissive spatial subportion, a blue-transmissive spatial subportion, a green-transmissive spatial subportion, and a SWIR-transmissive spatial subportion. In some emodiments, the filter array comprises: a first portion that transmits some or all of the reflected portion of the visible light illumination and blocks the reflected portion of the SWIR illumination, a second portion that transmits a first wavelength range of the reflected portion of the SWIR illumination, blocks a second wavelength range of the reflected portion of the SWIR illumination, and blocks the reflected portion of the visible light illumination, and a third portion that transmits the second wavelength range of the reflected portion of the SWIR illumination, blocks the first wavelength range of the reflected portion of the SWIR illumination, and blocks the reflected portion of the visible light illumination

The SWIR illumination may have a first polarization. The reflected portion of the SWIR illumination sensed by the sensor may have a second polarization opposite the first polarization. A polarizer may be arranged between the biological tissue and the sensor.

The sensor may include a stack of photosensors. The stack of photosensors can comprise a first photosensor configured to detect a blue portion of the reflected visible light illumination, a second photosensor configured to detect a green portion of the reflected visible light illumination, a third photosensor configured to detect a red portion of the reflected visible light illumination, and a fourth photosensor configured to detect the reflected portion of the SWIR illumination.

The biological tissue being imaged may include a first region having a first water content (e.g., volumetric density of water) and a second region having a second water content lower than the first water content. The biological tissue may be free of a contrast agent. The controller may be configured to generate the at least one image without reference light and/or without information from ambient light surrounding the sensor.

A provided method for imaging biological tissue may comprise providing, by one or more optical sources, short-wave infrared (SWIR) illumination and visible light illumination, sensing, by a sensor, a reflected portion of the SWIR illumination and a reflected portion of the visible light illumination, receiving, by a controller in communication with the sensor, from the sensor, first information corresponding to the reflected portion of the SWIR illumination, receiving, by the controller, from the sensor, second information corresponding to the reflected portion of the visible light illumination, generating, by the controller, at least one image of the biological tissue using the first information and the second information, and outputting, by the controller, the at least one image to at least one of a display and/or a memory.

A non-transitory computer-readable storage medium may store instructions for imaging biological tissue, the instructions configured to be executed by one or more processors of a system to cause the system to provide, by one or more optical sources, short-wave infrared (SWIR) illumination and visible light illumination, sense, by a sensor, a reflected portion of the SWIR illumination and a reflected portion of the visible light illumination, receive, by a controller in communication with the sensor, from the sensor, first information corresponding to the reflected portion of the SWIR illumination, receive, by the controller, from the sensor, second information corresponding to the reflected portion of the visible light illumination, generate, by the controller, at least one image of the biological tissue using the first information and the second information, and output, by the controller, the at least one image to at least one of a display and/or a memory.

### BRIEF DESCRIPTION OF THE FIGURES

The following figures show various systems and methods for single-sensor visible light and SWIR imaging. The systems and methods shown in the figures may have any one or more of the features described herein.

FIG. 1 shows an example system for single-sensor visible light and SWIR imaging, according to some embodiments.

FIG. 2 shows another example system for single-sensor visible light and SWIR imaging, according to some embodiments.

FIG. 3 shows another example system for single-sensor visible light and SWIR imaging, according to some embodiments.

FIG. 4A shows a filter array for a single-sensor visible light and SWIR imaging system, according to some embodiments.

FIG. 4B shows another filter array for a single-sensor visible light and SWIR imaging system, according to some embodiments.

FIG. 4C shows another filter array for a single-sensor visible light and SWIR imaging system, according to some embodiments.

FIG. 4D shows another filter array for a single-sensor visible light and SWIR imaging system, according to some embodiments.

FIG. 4E shows another filter array for a single-sensor visible light and SWIR imaging system, according to some embodiments.

FIG. 5 shows a sensor stack for a single-sensor visible light and SWIR imaging system, according to some embodiments.

FIG. 6 shows another example system for single-sensor visible light and SWIR imaging, according to some embodiments.

FIG. 7 shows a method for single-sensor visible light and SWIR imaging, according to some embodiments.

FIG. 8 shows a computer system, according to some embodiments.

### DETAILED DESCRIPTION

Provided are techniques for single-sensor, multi-wavelength imaging of biological tissue. The disclosed systems and methods may employ a single sensor to capture images of biological tissue in multiple wavelengths, for example to capture both short-wave infrared (SWIR) images and visible light images of the biological tissue. The captured images may be displayed, stored, and/or used to generate a composite image. Images produced using the described techniques may show target tissue (e.g., lymph nodes) and/or target features with high contrast, facilitating the identification and examination of said target tissue without requiring the use of contrast agents, without the use of reference light, and without information from ambient light surrounding the sensor.

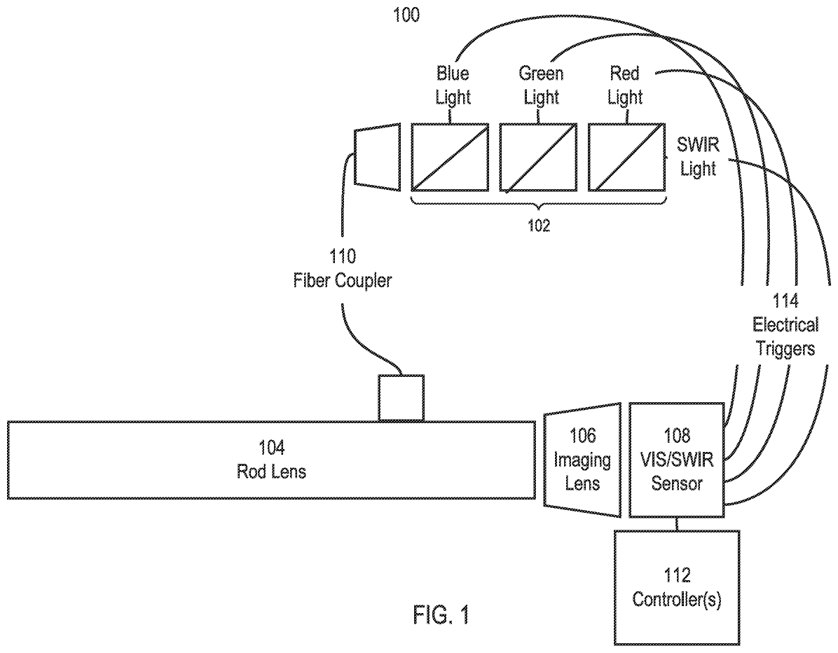

An exemplary imaging system 100 for SWIR and visible-light imaging of biological tissue is shown in FIG. 1. System 100 may include one or more light sources 102 configured to provide SWIR illumination and visible light illumination, one or more lenses (104, 106) for directing light to biological tissue and for collecting light reflected by biological tissue, and a sensor 108 for detecting light in the visible wavelength range and in the SWIR wavelength range. System 100 may be a component of a laparoscope.

Light source(s) 102 can be implemented using any suitable light-emitting devices, for example one or more laser light sources, one or more LED light sources, or combinations thereof. In some embodiments, light source(s) 102 is a single light source that emits in both the SWIR and visible light ranges. In other embodiments, light source(s) 102 comprises a first light source that emits in the SWIR range and a second set of one or more light sources that emit in the visible light range. Visible light illumination may be provided by multiple light sources that emit in different visible light wavelength ranges. For example, as shown in FIG. 1, visible light illumination may be provided by separate light sources that emit in the blue, green, and red visible light ranges. The SWIR illumination and visible light illumination may be delivered to and incident on the biological tissue to be imaged.

Light source(s) 102 may provide SWIR illumination in a wavelength range of about 800-2600 nm, 800-1700 nm, 900-2000 nm, 1000-2600 nm, 1000-1700 nm, 1500-1700 nm, 1500-2600 nm, or in any sub-portion of any one or more of said wavelength ranges. For example, light source(s) 102 may provide SWIR illumination with a wavelength of approximately 800 nm, 850 nm, 900 nm, 950 nm, 1000 nm, 1050 nm, 1100 nm, 1150 nm, 1200 nm, 1250 nm, 1300 nm, 1350 nm, 1400 nm, 1450 nm, 1500 nm, 1550 nm, 1600 nm, 1650 nm, 1700 nm, 1750 nm, 1800 nm, 1850 nm, 1900 nm, 1950 nm, 2000 nm, 2050 nm, 2100 nm, 2150 nm, 2200 nm, 2250 nm, 2300 nm, 2350 nm, 2400 nm, 2450 nm, 2500 nm, 2550 nm, or 2600 nm. In some embodiments, light source(s) 102 provide SWIR illumination at multiple SWIR wavelengths, for example SWIR illumination with a wavelength of approximately 800 nm, SWIR illumination with a wavelength of approximately 1700 nm, and SWIR illumination with a wavelength of approximately 2600 nm.

Visible light illumination may be provided by light source(s) in a wavelength range of about 380-750 nm, 450-625 nm, 485-590 nm, 500-565 nm, 450-750 nm, 500-750 nm, or 565-750 nm, or in any sub-portion of any one or more of said wavelength ranges. For example, light source(s) 102 may provide visible light illumination with a wavelength of approximately 450 nm, 455 nm, 460 nm, 465 nm, 470 nm, 475 nm, 480 nm, 485 nm, 490 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575 nm, 580 nm, 585 nm, 590 nm, 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm, 685 nm, 690 nm, 700 nm, 705 nm, 710 nm, 715 nm, 720 nm, 725 nm, 730 nm, 735 nm, 740 nm, 745 nm, or 750 nm. In some embodiments, light source(s) 102 provide visible light illumination at multiple visible wavelengths, for example visible light illumination with a wavelength of between 450 and 485 nm, visible light illumination with a wavelength between 500 and 565 nm, and visible light illumination with a wavelength of between 625 and 750 nm.

Rod lens 104 may deliver the SWIR illumination and visible light illumination provided by light source(s) 102 to the biological tissue to be imaged. Rod lens 104 may be a rod lens of the laparoscope of which system 100 is a component. Light source(s) 102 may transmit light to rod lens 104 through a fiber coupler 110 or any other suitable optical coupler.

Light that is reflected by the biological tissue to be imaged may be collected by rod lens 104 and subsequently guided to VIS/SWIR sensor 108 by imaging lens 106. The reflected light collected by system 100 may include both a reflected portion of the SWIR illumination and a reflected portion of the visible light illumination. The reflected portion of the SWIR illumination may be in any one or more of the same SWIR wavelength ranges recited above for the SWIR illumination, or in any sub-portion thereof. Likewise, the reflected portion of the visible light illumination may be in any one or more of the same visible light wavelength ranges recited above for the visible light illumination, or in any sub-portion thereof.

Sensor 108 may be disposed at a proximal end of the laparoscope behind imaging lens 106 and rod lens 104. Sensor 108 may detect some or all of the reflected SWIR light and may detect some or all of the reflected visible light. Sensor 108 may be sensitive to SWIR illumination in any one or more of the same SWIR wavelength ranges recited above for the SWIR illumination, or in any sub-portion thereof, and may be sensitive to visible light illumination in any one or more of the same visible light wavelength ranges recited above for the visible light illumination, or in any sub-portion thereof. In some embodiments, sensor 108 is monochrome in the visible light wavelength range; in other embodiments, sensor 108 is a color-sensitive sensor.

Sensor 108 may be any device or combination of devices configured to detect both SWIR light and visible light. In some embodiments, sensor 108 includes one or more cameras, for example a silicon camera, an InGaAs camera, a black silicon camera, a germanium camera, a germanium-tin on silicon camera, a quantum dot shortwave infrared camera, a mercury-cadmium-telluride camera, or a combination thereof. In some embodiments, sensor 108 comprises one or more color filter arrays or one or more photosensor stacks.

Light source(s) 102 and/or sensor 108 may be electronically coupled to and controlled by one or more controllers 112. Controller(s) 112 can be, e.g., a computer system such as a laptop, a tablet, a desktop computer, or a microcontroller. When sensor 108 detects reflected visible light and reflected SWIR light, information corresponding to the reflected visible light and the reflected SWIR light may be transmitted by sensor 108 to controller(s) 112. Controller(s) 112 may use the information received from sensor 108 to generate one or more images or one or more videos. The images/videos may be separate images/videos corresponding to one or more wavelength bands (e.g., separate visible light and SWIR images) or composite images/videos. Controller(s) 112 may then output the generated image(s) and/or video(s) to a display (e.g., a computer monitor) and/or a memory (e.g., a memory of controller(s) 112).

Controller(s) 112 may be configured to synchronize functionality between sensor 108 and light source(s) 102, for example through one or more electrical triggers 114 connecting light source(s) 102 and sensor 108. In some embodiments, controller(s) 112 may be configured to control light source(s) 102 to cause light source(s) 102 to emit the SWIR illumination and visible light illumination simultaneously. In other embodiments, controller(s) 112 may be configured to control light source(s) 102 to cause light source(s) 102 to emit the SWIR illumination and visible light illumination simultaneously in a pulsed timing scheme such that visible light illumination is emitted in alternating temporal pulses with SWIR illumination. When a pulsed timing scheme is used, controller 112 may synchronize sensor 108 with the pulses from light source(s) 102 such that SWIR image frames and visible light image frames are captured. In some embodiments, white light images with red, green, and blue illumination are captured; in other embodiments, images with illumination in a specific visible-light color are captured. The captured visible and SWIR image frames may be used (e.g., by controller(s) 112) to generate separate images or video and/or used to generate composite images or video.

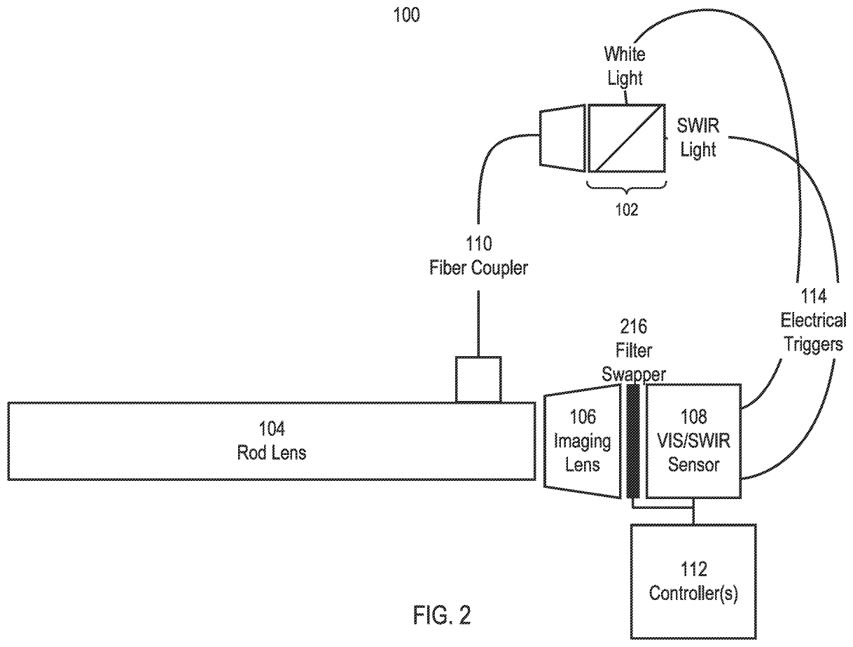

In embodiments in which light source(s) 102 provide continuous, simultaneous SWIR and visible light illumination, a filter wheel or other mechanical filter swapper 216 can be used to move one or more filters in an out of an optical path of sensor 108, as shown in FIG. 2. Filter swapper 216 may be disposed in front of sensor 108, for example between imaging lens 106 and sensor 108, and may be used to selectively block different wavelengths of reflected light. Controller(s) 112 may control movement of filter swapper 216 and may coordinate functionality of sensor 108 with movement of filter swapper 216 such that SWIR image frames and visible light image frames are captured. The captured frames may be used to generate separate images or video and/or used to generate composite images or video.

Filter swapper 216 may selectively position one or more of the following filters in the optical path of sensor 108: a SWIR-transmissive filter that transmits SWIR light and blocks visible light; a visible-transmissive filter that transmits visible light and blocks SWIR light; a red-transmissive filter that transmits red light and blocks blue, green, and SWIR light; a green-transmissive filter that transmits green light and blocks blue, red, and SWIR light; a blue-transmissive filter that transmits blue light and blocks green, red, and SWIR light; and a yellow-transmissive filter that transmits yellow light and blocks red, green, blue, and SWIR light.

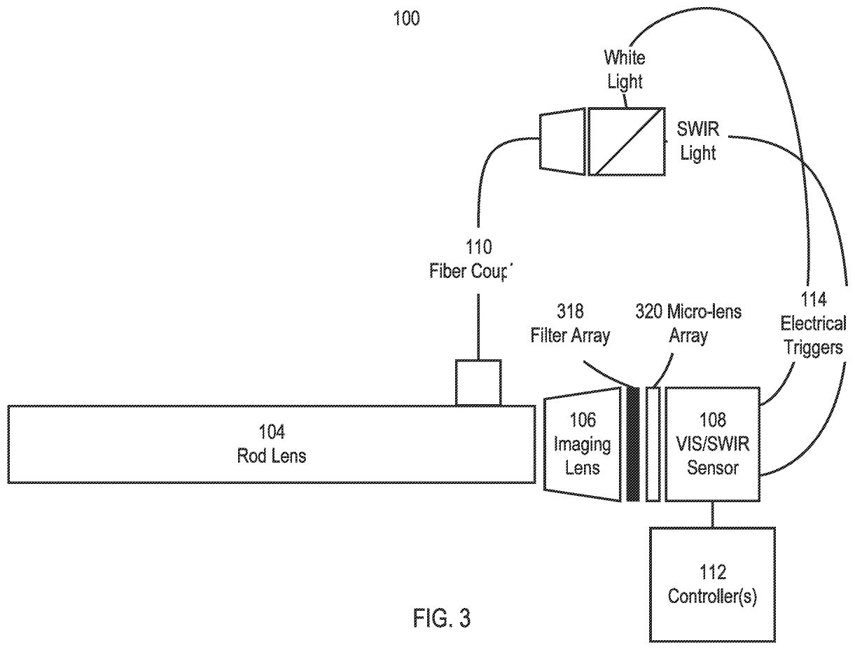

Alternatively, or in addition to, a filter wheel or mechanical filter swapper such as filter swapper 216, in embodiments in which light source(s) 102 provide continuous, simultaneous SWIR and visible light illumination, a color filter 318 can be used to select for light wavelengths that are allowed to be incident on sensor 108, as shown in FIG. 3.

Filter array 318 may be implemented using a visible and SWIR filter mosaic. The filter mosaic may allow for a spatial array of visible (e.g., blue, green, and red) and SWIR pixels to be captured based on simultaneous capture of reflected SWIR illumination and reflected visible light illumination. Any spatial arrangement of any combination of visible and SWIR color filters may make up the filter mosaic. Example filter mosaics include (but are not limited to) RGB (red, green, blue)+SWIR filter mosaics, RYB (red, yellow, blue)+SWIR filter mosaics, CYGM (cyan, yellow, green, magenta)+SWIR filter mosaics, RGBE (red, green, blue, emerald) filter mosaics, and RGBW (red, green, blue, white)+SWIR filter mosaics. In some embodiments, a filter mosaic may include multiple SWIR filters each configured to transmit a different SWIR wavelength band (e.g., a RGB (red, green, blue)+SWIR1+SWIR2 filter mosaic, where the SWIR1 filter(s) transmit SWIR light in a first SWIR wavelength band and the SWIR2 filter(s) transmit SWIR light in a second SWIR wavelength band). Additionally, a filter mosaic may have any dimension; for example, a filter mosaic may be a 2×2 array of filters, a 3×3 array of filters, a 4×4 array of filters, etc.

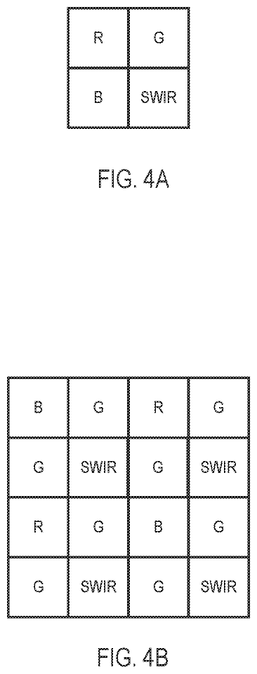

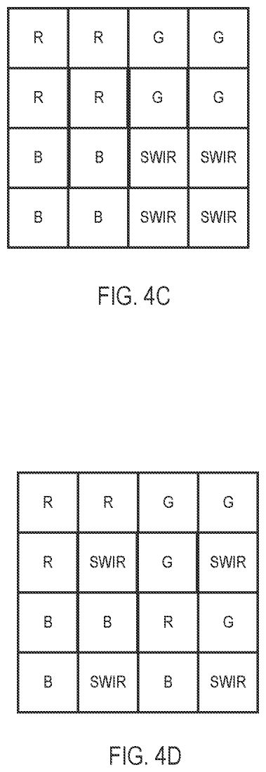

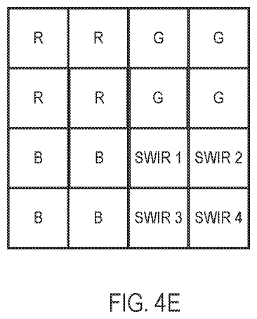

Diagrams of various example filter mosaics are provided in FIGS. 4A-4E. As shown, in some embodiments, a filter array comprises a first spatial portion that transmits some or all of the reflected portion of the visible light illumination and blocks the reflected portion of the SWIR illumination, and a second spatial portion that transmits some or all of the reflected portion of the SWIR illumination and blocks the reflected portion of the visible light illumination. For example, a filter array may comprise a first portion that transmits a green portion of the reflected visible light illumination while blocking blue and red portions of the reflected portion of the visible light illumination and blocking the reflected portion of the SWIR illumination, a second portion that transmits the blue portion of the reflected visible light illumination while blocking the green and red portions of the reflected portion of the visible light illumination and blocking the reflected portion of the SWIR illumination, a third portion that transmits the red portion of the reflected visible light illumination while blocking the blue and green portions of the reflected portion of the visible light illumination and blocking the reflected portion of the SWIR illumination, and a fourth portion that blocks the green, blue, and red portions of the reflected portion of the visible light illumination and transmits the reflected portion of the SWIR illumination.

The first, second, third, and fourth portions may be arranged in a repeating 2×2 tile arrangement in the filter array (e.g., as shown in FIG. 4A) or in a repeating 4×4 tile arrangement in the filter array (e.g., as shown in FIGS. 4B-4E). If the spatial portions are arranged in a repeating 4×4 tile arrangement, each repeating 4×4 tile portion in the repeating 4×4 tile arrangement may comprise a twice as many green-transmissive spatial portions as it does blue-transmissive spatial portions, red-transmissive spatial portions, or SWIR-transmissive spatial portions (e.g., as shown in FIG. 4B). The 4×4 tile portion may not include any laterally or vertically adjacent blue-transmissive portions, any laterally or vertically adjacent red-transmissive portions, any laterally or vertically adjacent green-transmissive portions, or any laterally or vertically adjacent SWIR-transmissive portions (e.g., as shown in FIG. 4B). In other embodiments, the 4×4 tile-arrangement comprises four 2×2 blocks, each 2×2 block comprising four spatial portions that are transmissive of a single respective wavelength range (e.g., as shown in FIG. 4C).

In other embodiments, the 4×4 tile-arrangement comprises four 2×2 blocks. A first 2×2 block may comprise three red-transmissive spatial subportions and one SWIR-transmissive spatial subportion. A second 2×2 block may three green-transmissive spatial subportions and one SWIR-transmissive spatial subportion. A third 2×2 block may comprise three blue-transmissive spatial subportions and one SWIR-transmissive spatial subportion. A fourth 2×2 block may comprise a red-transmissive spatial subportion, a blue-transmissive spatial subportion, a green-transmissive spatial subportion, and a SWIR-transmissive spatial subportion (e.g., as shown in FIG. 4D).

In some embodiments, the filter array comprises a first portion that transmits some or all of the reflected portion of the visible light illumination and blocks the reflected portion of the SWIR illumination, a second portion that transmits a first wavelength range of the reflected portion of the SWIR illumination, blocks a second wavelength range of the reflected portion of the SWIR illumination, and blocks the reflected portion of the visible light illumination, and a third portion that transmits the second wavelength range of the reflected portion of the SWIR illumination, blocks the first wavelength range of the reflected portion of the SWIR illumination, and blocks the reflected portion of the visible light illumination. For example, as shown in FIG. 4E, the filter array may comprise a first spatial portion that is red-transmissive, a second spatial portion that is green-transmissive, a third spatial portion that is blue-transmissive, a fourth spatial portion that is transmissive to a first wavelength range of the reflected portion of SWIR illumination (“SWIR1”), a fifth spatial portion that is transmissive to a second wavelength range of the reflected portion of SWIR illumination (“SWIR2”), a sixth spatial portion that is transmissive to a third wavelength range of the reflected portion of SWIR illumination (“SWIR3”), and a seventh spatial portion that is transmissive to a fourth wavelength range of the reflected portion of SWIR illumination (“SWIR4”).

In some embodiments, a micro-lens array may be disposed between filter array 318 and sensor 108.

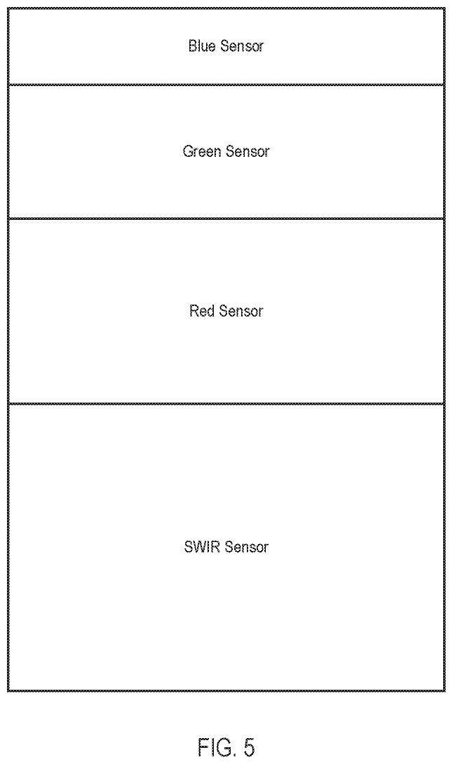

In some embodiments in which light source(s) 102 provide continuous, simultaneous SWIR and visible light illumination, sensor 108 may include a photosensor stack that includes two or more layers of photosensors. For instance, a sensor stack may be a blue+green+red+SWIR sensor stack, a blue+yellow+red+SWIR sensor stack, a cyan+yellow+green+magenta+SWIR sensor stack, a red+green+blue+emerald sensor stack, a red+green+SWIR sensor stack, or a blue+green+SWIR sensor stack. An example sensor stack is illustrated in FIG. 5. Light throughput to sensor 108 may be higher when a sensor stack, rather than a filter array, is used; accordingly, embodiments of system 100 wherein sensor 108 comprises a sensor stack may be ideal for imaging biological tissue in low-light situations.

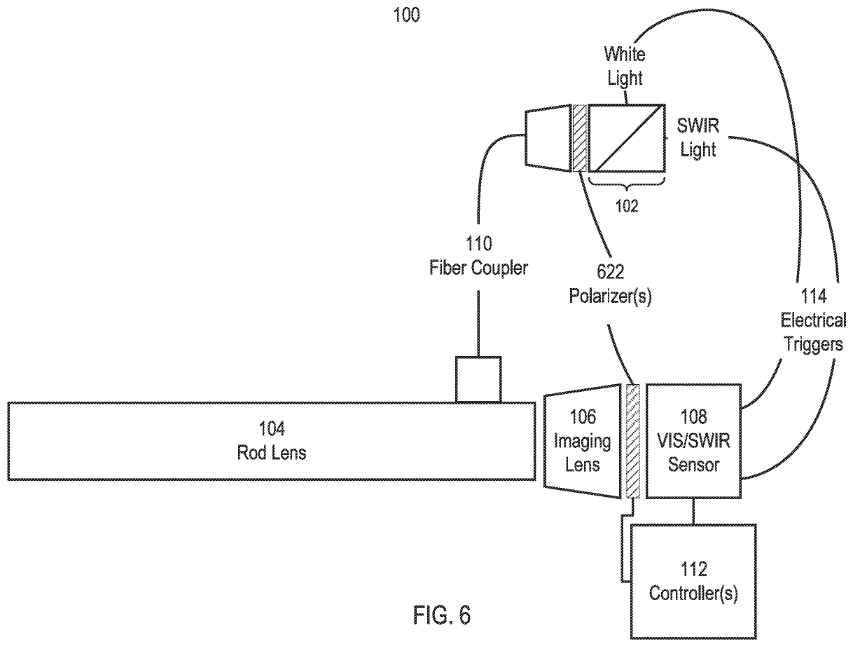

In some embodiments, light emitted by light source(s) 102 is polarized. For example, the SWIR illumination may have a first polarization, and the reflected portion of the SWIR illumination that is detected by the sensor may have a second polarization that is opposite the first polarization. Cross-polarization imaging modalities may be used. One or more polarizers 622 in the optical path of system may polarize the illumination light and/or the reflected portion of the illumination light, e.g., as illustrated in FIG. 6.

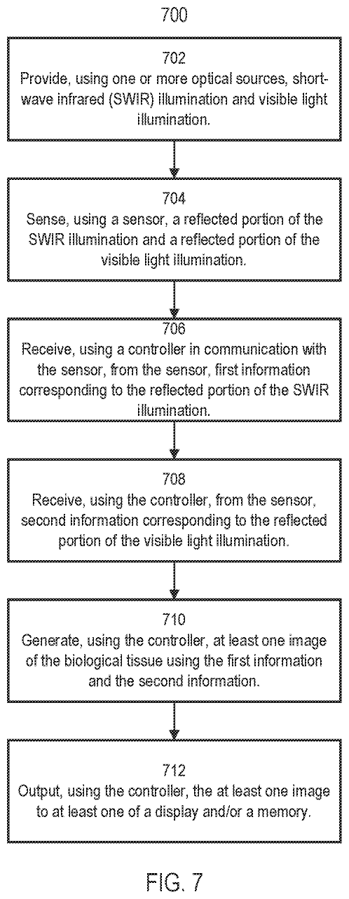

An exemplary method 700 for single-sensor visible light and SWIR imaging biological tissue is provided in FIG. 7. Method 700 may be executed using a system for imaging biological tissue using visible and SWIR light, for example any of the embodiments of system 100 shown in FIGS. 1-3 and 6. The biological tissue that is imaged using method 700 may include one or more different types or biological tissue and/or one or more different regions of biological tissue. In some embodiments, the biological tissue may include a lymphatic component.

In some embodiments, the biological tissue may include one or more different types or biological tissue and/or one or more different regions of biological tissue having different water content. Because water is absorptive in the SWIR range (e.g., around 1550 nm), imaging using SWIR illumination may be effective at differentiating biological tissue with higher water content from biological tissue with lower water content. For example, lymph nodes which are high in water content may be differentiated from fat which is low in water content.

To image the biological tissue, SWIR illumination and visible light illumination may be provided to the biological tissue by one or more optical sources (e.g., light source(s) 102 shown in FIGS. 1-3) (step 702 of method 700). The SWIR illumination and visible light illumination may be delivered to the biological tissue by a lens such as a rod lens of a laparoscope (e.g., rod lens 104 shown in FIGS. 1-3 and 6). A reflected portion of the SWIR illumination and a reflected portion of the visible light illumination may then be sensed using a sensor (e.g., sensor 108 shown in FIGS. 1-3 and 6) (step 704 of method 700). The reflected portion of the SWIR illumination and the reflected portion of the visible light illumination may be guided to sensor from the biological tissue by one or more lenses (e.g., rod lens 104 and/or imaging lens 106 shown in FIGS. 1-3 and 6). The sensor may transmit first information corresponding to the reflected portion of the SWIR illumination and second information corresponding to the reflected portion of the visible light illumination to a controller (e.g., controller(s) 112 shown in FIGS. 1-3 and 6) in communication with the sensor (steps 706-708 of method 700). Using the first information and the second information, the controller may generate at least one image of the biological tissue (step 710 of method 700). The controller may then output the at least one image to at least one of a display and/or a memory (step 712 of method 700).

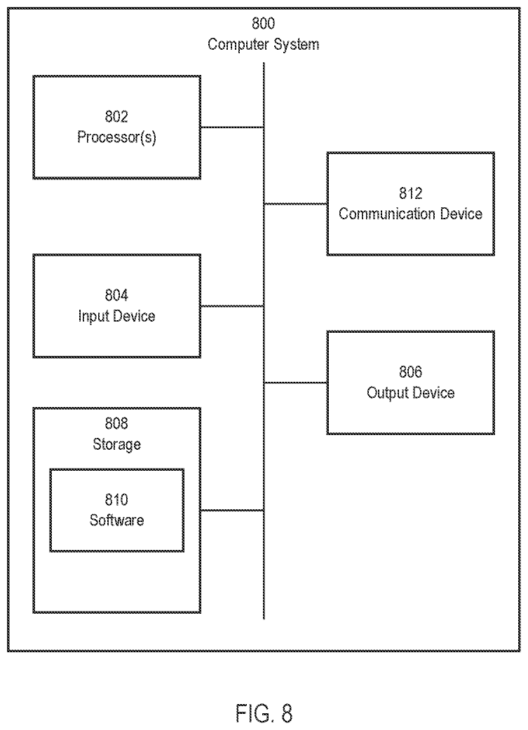

FIG. 8 shows an exemplary computer system 800 that may be used to generate images and/or video of biological tissue based on information received from a sensor in the provided visible and SWIR imaging systems (e.g., system 100). In other words, computer system 800 may be used to implement a controller in a system for imaging biological tissue using visible and SWIR light such as system 100. Computer system 800 can be any suitable type of microprocessor-based device, such as a personal computer, workstation, server, or handheld computing device (portable electronic device) such as a phone or tablet, or dedicated device. As shown in FIG. 8, computer system 800 may include one or more processors 802, an input device 804, an output device 806, storage 808 storing software 810, and a communication device 812.

Input device 804 and output device 806 can be connectable or integrated with system 102. Input device 804 may be any suitable device that provides input, such as a touch screen, keyboard or keypad, mouse, or voice-recognition device. Likewise, output device 806 can be any suitable device that provides output, such as a display, touch screen, haptics device, or speaker.

Storage 808 can be any suitable device that provides storage, such as an electrical, magnetic, or optical memory, including a RAM, cache, hard drive, removable storage disk, or other non-transitory computer readable medium. Communication device 812 can include any suitable device capable of transmitting and receiving signals over a network, such as a network interface chip or device. The components of computer system 800 can be connected in any suitable manner, such as via a physical bus or via a wireless network.

Processor(s) 802 may be or comprise any suitable processor or combination of processors, including any of, or any combination of, a central processing unit (CPU), a field programmable gate array (FPGA), and an application-specific integrated circuit (ASIC). Software 810, which can be stored in storage 808 and executed by processor(s) 802, can include, for example, the programming that embodies the functionality of the present disclosure. Software 810 may be stored and/or transported within any non-transitory computer-readable storage medium for use by or in connection with an instruction execution system, apparatus, or device that can fetch instructions associated with the software from the instruction execution system, apparatus, or device and execute the instructions. In the context of this disclosure, a computer-readable storage medium can be any medium, such as storage 808, that can contain or store programming for use by or in connection with an instruction execution system, apparatus, or device.

Software 810 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Computer system 800 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computer system 800 can implement any operating system suitable for operating on the network. Software 810 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The foregoing description, for the purpose of explanation, has been described with reference to specific embodiments and/or examples. However, the illustrative discussions above are not intended to be exhaustive or to limit the invention to the precise forms disclosed. Many modifications and variations are possible in view of the above teachings.

The embodiments were chosen and described in order to best explain the principles of the techniques and their practical applications. Others skilled in the art are thereby enabled to best utilize the techniques and various embodiments with various modifications as are suited to the particular use contemplated.

As used herein, the singular forms “a”, “an”, and “the” include the plural reference unless the context clearly dictates otherwise. Reference to “about” a value or parameter or “approximately” a value or parameter herein includes (and describes) variations that are directed to that value or parameter per se. For example, description referring to “about X” includes description of “X”. It is understood that aspects and variations of the invention described herein include “consisting of” and/or “consisting essentially of” aspects and variations.

When a range of values or values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

Any of the systems, methods, techniques, and/or features disclosed herein may be combined, in whole or in part, with any other systems, methods, techniques, and/or features disclosed herein.

1. A system for imaging biological tissue, the system comprising:
one or more optical sources configured to provide short-wave infrared (SWIR) illumination and to provide visible light illumination;
a sensor configured to sense a reflected portion of the SWIR illumination and a reflected portion of the visible light illumination; and
a controller in communication with the sensor and configured to:
receive, from the sensor, first information corresponding to the reflected portion of the SWIR illumination;
receive, from the sensor, second information corresponding to the reflected portion of the visible light illumination;
generate at least one image of the biological tissue using the first information and the second information; and
output the at least one image to at least one of a display and/or a memory.

2. The system of claim 1, wherein the one or more optical sources are configured to alternately provide the SWIR illumination and the visible light illumination according to a temporal pulsing scheme.

3. The system of claim 1, wherein the one or more optical sources comprise a first optical source configured to provide the SWIR illumination and a second optical source configured to provide the visible light illumination.

4. The system of claim 1, wherein the one or more optical sources comprise a first optical source configured to provide the SWIR illumination, a second optical source configured to provide red light illumination, a third optical source configured to provide green light illumination, and a fourth optical source configured to provide blue light illumination.

5. The system of claim 1, further comprising:
a first filter configured to block at least some of the SWIR illumination; and
a second filter configured to block at least some of the visible light illumination.

6. The system of claim 5, further comprising a filter support device configured to move at least one of the first filter and the second filter between a first position in an optical path of the system to a second position outside the optical path of the system.

7. The system of claim 1, wherein the one or more optical sources are configured to provide the SWIR illumination and the visible light illumination simultaneously.

8. The system of claim 1, further comprising a filter array configured to spatially selectively block and transmit the reflected portion of the SWIR illumination and the reflected portion of the visible light illumination.

9. The system of claim 8, wherein the filter array comprises a first portion that transmits some or all of the reflected portion of the visible light illumination and blocks the reflected portion of the SWIR illumination, and a second portion that transmits some or all of the reflected portion of the SWIR illumination and blocks the reflected portion of the visible light illumination.

10. The system of claim 8, wherein the filter array comprises:
a first portion that transmits a green portion of the reflected visible light illumination while blocking blue and red portions of the reflected portion of the visible light illumination and blocking the reflected portion of the SWIR illumination;
a second portion that transmits the blue portion of the reflected visible light illumination while blocking the green and red portions of the reflected portion of the visible light illumination and blocking the reflected portion of the SWIR illumination;
a third portion that transmits the red portion of the reflected visible light illumination while blocking the blue and green portions of the reflected portion of the visible light illumination and blocking the reflected portion of the SWIR illumination; and
a fourth portion that blocks the green, blue, and red portions of the reflected portion of the visible light illumination and transmits the reflected portion of the SWIR illumination.

11. The system of claim 10, wherein the first, second, third, and fourth portions are arranged in a repeating 2×2 tile arrangement in the filter array.

12. The system of claim 10, wherein the first, second, third, and fourth portions are arranged in a repeating 4×4 tile arrangement in the filter array,

13. The system of claim 12, wherein each repeating 4×4 tile portion in the repeating 4×4 tile arrangement comprises a twice as many green-transmissive spatial portions as it does blue-transmissive spatial portions, red-transmissive spatial portions, or SWIR-transmissive spatial portions.

14. The system of claim 12, wherein the 4×4 tile portion does not include any laterally or vertically adjacent blue-transmissive portions, any laterally or vertically adjacent red-transmissive portions, any laterally or vertically adjacent green-transmissive portions, or any laterally or vertically adjacent SWIR-transmissive portions.

15. The system of claim 12, wherein the 4×4 tile-arrangement comprises four 2×2 blocks, each 2×2 block comprising four spatial portions that are transmissive of a single respective wavelength range.

16. The system of claim 12, wherein:
the 4×4 tile-arrangement comprises four 2×2 blocks;
a first 2×2 block comprises three red-transmissive spatial subportions and one SWIR-transmissive spatial subportion;
a second 2×2 block comprises three green-transmissive spatial subportions and one SWIR-transmissive spatial subportion;
a third 2×2 block comprises three blue-transmissive spatial subportions and one SWIR-transmissive spatial subportion; and
a fourth 2×2 block comprises a red-transmissive spatial subportion, a blue-transmissive spatial subportion, a green-transmissive spatial subportion, and a SWIR-transmissive spatial subportion.

17. The system of claim 8, wherein the filter array comprises:
a first portion that transmits some or all of the reflected portion of the visible light illumination and blocks the reflected portion of the SWIR illumination,
a second portion that transmits a first wavelength range of the reflected portion of the SWIR illumination, blocks a second wavelength range of the reflected portion of the SWIR illumination, and blocks the reflected portion of the visible light illumination; and
a third portion that transmits the second wavelength range of the reflected portion of the SWIR illumination, blocks the first wavelength range of the reflected portion of the SWIR illumination, and blocks the reflected portion of the visible light illumination.

18. The system of claim 1, wherein the biological tissue comprises a first region having a first water content and a second region having a second water content lower than the first water content.

19. The system of claim 1, wherein the one or more optical sources comprise a laser.

20. The system of claim 1, wherein the one or more optical sources comprise a light emitting diode.

21. The system of claim 1, wherein the sensor comprises one or more cameras selected from the following group: a silicon camera, an InGaAs camera, a black silicon camera, a germanium camera, a germanium-tin on silicon camera, a quantum dot shortwave infrared camera, and a mercury-cadmium-telluride camera.

22. The system of claim 1, wherein the SWIR illumination has a first polarization.

23. The system of claim 22, wherein the reflected portion of the SWIR illumination sensed by the sensor has a second polarization opposite the first polarization.

24. The system of claim 1, comprising a polarizer arranged between the biological tissue and the sensor.

25. The system of claim 1, wherein the biological tissue is free of a contrast agent.

26. The system of claim 1, wherein the controller is configured to generate the at least one image without reference light.

27. The system of claim 1, wherein the controller is configured to generate the at least one image without information from ambient light surrounding the sensor.

28. The system of claim 1, wherein the sensor comprises a stack of photosensors.

29. The system of claim 28, wherein the stack of photosensors comprises:
a first photosensor configured to detect a blue portion of the reflected visible light illumination;
a second photosensor configured to detect a green portion of the reflected visible light illumination;
a third photosensor configured to detect a red portion of the reflected visible light illumination; and
a fourth photosensor configured to detect the reflected portion of the SWIR illumination.

30. A method for imaging biological tissue, the method comprising:
providing, by one or more optical sources, short-wave infrared (SWIR) illumination and visible light illumination;
sensing, by a sensor, a reflected portion of the SWIR illumination and a reflected portion of the visible light illumination;
receiving, by a controller in communication with the sensor, from the sensor, first information corresponding to the reflected portion of the SWIR illumination;
receiving, by the controller, from the sensor, second information corresponding to the reflected portion of the visible light illumination;
generating, by the controller, at least one image of the biological tissue using the first information and the second information; and
outputting, by the controller, the at least one image to at least one of a display and/or a memory.

31. A non-transitory computer-readable storage medium storing instructions for imaging biological tissue, the instructions configured to be executed by one or more processors of a system to cause the system to:
provide, by one or more optical sources, short-wave infrared (SWIR) illumination and visible light illumination;
sense, by a sensor, a reflected portion of the SWIR illumination and a reflected portion of the visible light illumination;
receive, by a controller in communication with the sensor, from the sensor, first information corresponding to the reflected portion of the SWIR illumination;
receive, by the controller, from the sensor, second information corresponding to the reflected portion of the visible light illumination;
generate, by the controller, at least one image of the biological tissue using the first information and the second information; and
output, by the controller, the at least one image to at least one of a display and/or a memory.
